(19) United States
(12) Patent Application Publication
(10) Pub. No.: US 2003/0139650 A1
(43) Pub. Date: Jul. 24, 2003

(54) Endoscope

(76) Inventor: Hiroyuki Homma, Tokyo (JP)

Correspondence Address: Arnold International, P.O. BOX 129, Great Falls, VA, 22066, US

(21) Appl. No.: 10/320,502

(22) Filed: Dec. 17, 2002

(30) Foreign Application Priority Data
Jan. 18, 2002 (JP) 2002-010007

(51) Int. Cl. A61B 1/06

(52) U.S. Cl. 600/181; 600/178

(57) ABSTRACT

An endoscope is disclosed that includes a light source unit for illuminating an object, and an optical system which forms images of the object and includes a spectral filter. The spectral filter includes a first region which has a first spectral transmission and a second region which is peripheral to the first region and which has a second spectral transmission that is different from the first spectral transmission, to thereby enable endoscope images of the object to be obtained wherein fine details as carried by high spatial frequencies in the image light of certain wavelengths are emphasized for those wavelengths that are passed by the second region of the spectral filter. In addition, the endoscope may contain a phase mask and an image processing means which serve to extend the depth of field in the wavelength range passed by the second region of the spectral filter.

### BACKGROUND OF THE INVENTION

Conventionally, endoscopes obtain images inside a coelom by illuminating a part to be viewed. In this kind of endoscope, an electronic endoscope is used which guides illumination light from a light source into a coelom using a light guide, etc., and the reflected light is then imaged onto an image sensor. By processing the signals from the image sensor with a video processor, an endoscope image is displayed on an observation monitor and observed regions such as affected parts may be observed.

When observing ordinary living tissue with an endoscope, white light in the visible region is emitted by a light source unit and is then separated into beams of three different colors which are sequentially irradiated onto an object. The three beams of different colors are usually formed by passing white light through a rotating filter wheel having filters which pass, for example, red (R), green (G), and blue (B) wavelengths. The reflected light from the object is then synchronized and the images processed by a video processor in order to obtain a color image. Alternatively, a color image sensing device (herein termed a color sensing chip) may be placed at the image plane of an endoscope. White illuminating light that is reflected from an object is detected according to color components that are transmitted onto different detecting areas of the color sensing chip, and the image data is then processed by a video processor in order to obtain a color image.

The image necessary for performing observation and processing by an endoscope is preferably one that is optimized for diagnosis rather than being a natural image as viewed with human eyes. The invasion depth of light in the depth direction of tissues inside a coelom depends on the wavelength of the light. Visible light of short wavelengths, such as blue colors, reaches only the vicinity of the surface layer, due to it being absorbed and scattered by living tissue up to that depth, and the light that emerges from the surface is then observed.

Green light reaches deeper than the range where blue light reaches, and thus green light is absorbed and scattered to a deeper depth. Some of the green light that is scattered emerges from the surface and is then observed. Red light, due to its still longer wavelengths, reaches an even deeper range.

As shown in FIG. 5, tissues inside a coelom 51 often have a distribution of structures which absorb different colors of light with different efficiencies. An example of such tissues are blood vessels, which absorb different wavelengths of light with different efficiencies depending on the depth of the blood vessel. This is due to the blood vessel structures varying with depth, there being many capillary vessels 52 distributed near the surface of the mucosa layer, with thicker blood vessels 53 than the capillary vessels 52 being in a middle layer that lies just below the mucosa layer, and even thicker blood vessels 54 being distributed in an even deeper layer.

The endoscope described in Japanese Laid Open Patent Application 2001-88256 is an example of optimizing the spectral distribution of a light source unit depending on the properties of a living body. In that published patent application, diagnosis is made by obtaining deep layer tissue information of a desired living tissue using narrow-band light beams that are sequentially irradiated and are spectrally discrete (meaning the wavelength ranges in the different beams do not overlap one another).

Rather than using reflected light, there is an endoscope technology for observing images of living body structures using fluorescence. For example, Japanese Laid Open Patent Application No. 2001-198079 discloses an endoscope which detects auto-fluorescence from a living body, or which detects fluorescence of a chemical substance that has been injected into a living body, as a two-dimensional image. The image is then diagnosed to determine the extent of diseased tissue, such as from cancer. This technique utilizes the fact that, when excitation light having wavelengths in the range of 420 nm-480 nm is irradiated onto living cells, normal parts of living tissue emit significantly stronger fluorescent light at green wavelengths than do cancerous tumors. Thus, observations are made which rely on the brightness of the signals at green wavelengths in order to discern the extent of the diseased tissue.

Furthermore, it is known to optimize other aspects of an endoscope optical system. For example, Japanese Laid Open Patent Application 2000-5127 (which corresponds to U.S. Pat. No. 6,241,656) discloses an endoscope system having an extended depth of field. The method of extending the depth of field of the imaging optical system of this patent application was disclosed in Japanese Patent Publication H11-500235 (which corresponds to U.S. Pat. No. 5,748,371, hereby incorporated by reference). As shown in FIG. 11, this apparatus includes an optical phase mask in the light path between an object and its image on an image sensor as formed by an objective optical system. The optical phase mask is placed at a pupil position of the objective optical system, and an image processing device is used to construct the image based on data detected by the image sensor. In an ordinary imaging optical system which does not have such an optical phase mask, as the position of the object deviates from the in-focus position in two incremental steps in a single direction, the optical transfer function (which is shown in FIG. 13 for the in-focus position) first changes to that shown in FIG. 14, and then changes to that illustrated in FIG. 15. On the other hand, in a depth of field expansion optical system which employs an optical phase mask, the optical transfer function intensity distribution does not change much for the same corresponding in-focus and normally out-of-focus positions, as illustrated in FIGS. 16-18, respectively.

Referring to FIGS. 13-22, in each figure, the vertical axis is the optical transfer function, and the horizontal axis is the relative spatial frequency at the image plane, with the numerical value of “2” on the horizontal axis corresponding to the Nyquist frequency of the image sensor. Each image formed by the optical system is processed, using a reverse filter whose property is shown in FIG. 19, by an image processing device. In this manner the optical transfer function intensity distributions as shown in FIGS. 20-22 are obtained. These curves correspond in shape to the optical transfer function intensity distributions as shown in FIGS. 16-18, respectively, which are the curves obtained when the optical system is in focus.

Although natural images with a good color reproducibility can be obtained using an endoscope, when observing tissues in a coelom using an endoscope, there is ordinarily a problem in that information concerning tissues at a particular depth in a living body is mixed with information concerning tissues at other depths, thereby reducing the contrast in the image. This reduction in contrast occurs because light of different wavelengths, in the situation where light of different wavelength bands that overlap each other sequentially illuminates the object, is mixed uniformly.

Also, although desired deep layer tissue information can be obtained through observations with an endoscope using narrow-band, sequential light beams having discrete spectral properties that do not overlap, because the wavelength range of the illumination is narrow, there is a problem in that the obtained images are dark as compared with images that are obtained using sequential light beams, such as red, green, and blue beams that have overlapping wavelengths. Moreover, using overlapping wavelengths of red, green and blue for the sequential beams is much more suitable for good color reproduction. There is also a problem in that the reflected green light and especially the reflected blue light have narrow depths of field relative to that for red light. This is especially a problem where the object has fine details, since these result in the image having a high spatial frequency content.

As previously mentioned, in observations using narrow-band, sequential light beams with discrete spectral properties, there is a problem of insufficient brightness due to a decrease of transmitted light resulting from the pass-band wavelength ranges being narrow. And, in observations that use fluorescence instead of reflected light, there is a problem of insufficient brightness because the fluorescence is faint. If the F-number of the objective optical system is reduced in order to increase the image brightness, this causes the depth of field to become more narrow. As a result, in places such as the esophagus where violent motions may occur, it is difficult to maintain a surface of interest in focus, since the observation distance can change rapidly. In such a situation, it is required that the depth of field be broadened. And, it is desired that fluorescent light images have a good contrast so as to reveal fine details of an object.

### BRIEF SUMMARY OF THE INVENTION

The objects of the present invention are to provide: (a) an endoscope which can observe deep tissues of a living body with good contrast; (b) an endoscope where observations that employ reflected light have a wide depth of field, even in the case where the images are formed using green or blue wavelengths of light, (c) an endoscope where a bright image with sufficient depth of field can be obtained even when employing narrow-band light beams which sequentially irradiate an object of interest with light having discrete spectral properties, and (d) an endoscope where a bright image of any desired depth of field can be obtained even when observing using fluorescent light.

### BRIEF DESCRIPTION OF THE DRAWINGS

The present invention will become more fully understood from the detailed description given below and the accompanying drawings, which are given by way of illustration only and thus are not limitative of the present invention, wherein:

FIG. 1 is a block diagram of the configuration of an endoscope according to Embodiment 1 of the present invention;

FIG. 2 shows the configuration of a rotating filter wheel of the invention;

FIG. 3 shows the optical properties of a first filter set of the invention, wherein the passbands of three filters somewhat overlap;

FIG. 4 shows the optical properties of a second filter set of the invention, wherein the passbands of three filters do not overlap;

FIG. 5 shows the structure of living body tissue which varies with depth beneath the tissue surface;

FIG. 6 illustrates how the color of scattered light from living tissue varies with the depth of the scattering beneath the tissue surface;

FIGS. 7(a)-7(c) show respective images using light of partially overlapping wavelength bands which are transmitted by the first filter set illustrated in FIG. 3;

FIGS. 8(a)-8(c) show respective images using light of the discrete wavelength bands which are transmitted by the second filter set illustrated in FIG. 4;

FIG. 9 shows a central region “a” and a peripheral region “b” of a spectral transmittance filter which, preferably, is placed in a light beam immediately following a brightness diaphragm which serves as a pupil in the imaging optical system of the invention;

FIGS. 10(a) and 10(b) show spectral properties of light beams, with FIG. 10(a) showing the spectral properties of light which has passed through the region “a” illustrated in FIG. 9, and FIG. 10(b) showing the spectral properties of light which has passed through the region “b” illustrated in FIG. 9;

FIG. 11 shows the configuration of an expanded depth of field optical system used with the present invention, which configuration corresponds to that taught in the prior art;

FIG. 12 is a perspective view to show the appearance of the mask illustrated in FIG. 11. The mask is an optical phase mask having a thickness which varies with X-Y position as illustrated, and which functions as a spatial frequency characteristic converter so as to cause the optical transfer function of the objective optical system of FIG. 11 to remain essentially constant within a range of in-focus position;

FIG. 13 shows the intensity profile of the optical transfer function when an object is at the focal point in a general optical system;

FIG. 14 is a graphical presentation to show the intensity profile of the optical transfer function when an object is a specified distance away from the focal point in a general optical system;

FIG. 15 is a graphical presentation to show the intensity profile of the optical transfer function when an object is farther away from the focal point than in FIG. 14 in a general optical system;

FIG. 16 is a graphical presentation to show the intensity profile of the optical transfer function when an object is at the focal point in an optical system having an extended depth of field;

FIG. 17 is a graphical presentation to show the intensity profile of the optical transfer function when an object is a specified distance away from the focal point in an optical system having an extended depth of field;

FIG. 18 is a graphical presentation to show the intensity profile of the optical transfer function when an object is farther away from the focal point than in FIG. 17, in an optical system having an extended depth of field;

FIG. 19 is a graphical presentation to show the characteristic of an inverse filter for processing the intensity profile of the optical transfer function in an optical system having an extended depth of field;

FIG. 20 is a graphical presentation to show the intensity profile of the optical transfer function after the intensity profile of the optical transfer function of FIG. 16 is processed using the inverse filter having the characteristic of FIG. 19;

FIG. 21 is a graphical presentation to show the intensity profile of the optical transfer function after the intensity profile of the optical transfer function of FIG. 17 is processed using the inverse filter having the characteristic of FIG. 19;

FIG. 22 is a graphical presentation to show the intensity profile of the optical transfer function after the intensity profile of the optical transfer function of FIG. 18 is processed using the inverse filter having the characteristic of FIG. 19;

FIG. 23 is a cross-sectional view of the imaging optical system according to Embodiment 1 of the invention;

FIG. 24 is a cross-sectional view of the imaging optical system according to Embodiment 2 of the invention;

FIG. 25 shows the spectral transmittance (i.e., transmittance versus wavelength) of the region “b” where the pupil is enlarged for certain wavelengths in the imaging optical system in Embodiment 3 of the invention;

FIG. 26 shows the spectral transmittance of the region “b” where the pupil is enlarged in the imaging optical system in Embodiment 4 of the invention;

FIGS. 27(a)-27(c) show the spectral transmittance of the blue, green and red transmission filters, respectively, of the second filter set of filters according to Embodiment 7 of the invention;

FIG. 28 shows the intensities of reflected light and fluorescent light from an object when illuminated with the filter set having the properties shown in FIGS. 27(a)-27(c);

FIG. 29 shows the spectral transmittance of the sets of color filters that form an array pattern on a color sensing chip according to Embodiment 9 of the invention;

FIG. 30 is a block diagram of the configuration of an endoscope according to Embodiment 9 of the invention;

FIG. 31 shows the spectral transmittance of the region “b” where the pupil is enlarged in the imaging optical system according to Embodiment 5 of the invention;

FIG. 32 shows the spectral transmittance of the region “b” where the pupil is enlarged in the imaging optical system according to Embodiment 6 of the invention;

FIG. 33 shows the spectral transmittance of the region “b” where the pupil is enlarged in the imaging optical system according to Embodiment 8 of the invention; and

FIG. 34 is an illustration for explaining depth of field.

### DETAILED DESCRIPTION

The present invention relates to medical-use endoscopes and industrial-use endoscopes. If an optical member such as a spectral filter which has a spectral transmittance distribution which transmits a higher proportion of light in a wavelength band where higher contrast is desired is placed in the vicinity where a pupil of an imaging optical system is enlarged, information of a specific wavelength band (i.e., the amount of light at those wavelengths) that contributes to an image will increase as compared with other wavelength bands. Therefore, the contrast of the wavelength band where higher contrast is desired will increase.

By this means, almost irrespective of the particular spectral output of a light source unit, it becomes possible to make an endoscopic observation having an emphasis on information at a particular wavelength band so as to yield a higher contrast. As is known, if an imaging optical system has a small F-number so as to yield a bright image, this will normally result in the depth of field being narrow. If, however, a spatial frequency characteristic conversion is performed by using, for example, an optical phase mask in conjunction with a signal processing unit (as is known in the art) so as to restore the spatial frequency content, an extended depth of field can be achieved for deep tissue information whose contrast has been selectively increased. Thus, according to the present invention, an endoscope having an emphasis at a particular wavelength band, as well as an expanded depth of field, can be provided. The expanded depth of field can be within a range of about 2 mm to 100 mm for an observed object. In an endoscope for medical use wherein, for example, one checks for a lesion inside a coelom during a screening and, should such a lesion be found, the endoscope is used to obtain a tissue sample, the expanded depth of field will normally be in the range of about 10 mm-100 mm. However, in order to observe the lesion with magnification and to obtain more detailed tissue information, the expanded depth of field may be set in a range of about 2 mm-30 mm. Also, in an endoscope that is to be used to perform tasks, an even larger depth of field may be set, such as from about 3 mm-80 mm.

In an endoscope which is used to perform observations with light of a specific wavelength, such as fluorescent light observations, the lesion part may need to be magnified in order to specify the boundary between a normal portion and a lesion portion of a living body, while still providing sufficient depth of field so as to project an image of the entire lesion part so as to determine to what degree the lesion part has infiltrated the living body tissue. For this purpose, the expanded depth of field may be set in a range from about 2 mm-50 mm. In this way, the expanded depth of field of an endoscope is set appropriately according to the use of the endoscope within the range of about 2 mm-100 mm to the observed object. Thus, the feature of the present invention with regard to emphasizing a particular spectral frequency band in order to provide better contrast to an image of interest can be applied.

A description will now be given regarding what is termed “depth of field”, using FIG. 34. When the image I of an object O is formed by an imaging optical system 60, a focused image is formed on a sensor surface of the CCD in the position of image I. If the object O is placed at the position O′, which is a distance Xn from the imaging optical system 60, the image position is formed at the position I′, which deviates from the I position. Conversely, if the object O is moved farther to the position O″, which is at a distance Xf from the imaging optical system 60, the image position will be at the position I″. If the position of the CCD is fixed, the images I′ and I″ at the CCD position become a blur circle of diameter δ, and the image becomes de-focused. However, if the CCD resolution is larger than the blur circle of diameter δ, the object appears to be in focus when it is within a position from O′ to O″, which corresponds to the distance D=Xf−Xn. This range D is called the depth of field. When the effective F-number of the optical system is F_{NO EFF}, and the focal distance is f_{L}, then the depth of field D is given by:

D=|1/Xn−1/Xf|=2 δFno_{EFF}/f_{L}².

In order to diagnose precisely how much a tumor has spread in a living body tissue (i.e., the range of a lesion), a very effective diagnosing method is to examine in detail the blood vessel structure running in the depth direction of a near-surface layer of a living body tissue using blue light and green light wavelength ranges.

In an endoscope observation, if light that forms an image of an object (such as coelom tissue), is to carry fine details of the object, then the pupil of the imaging optical system must be sufficiently large so as pass the high spatial frequency components of the image, which physically lie a distance from the optical axis that increases with increased spatial frequency. In the present invention, the pupil is enlarged for those wavelengths of specific interest to an observer of coelom tissue, and the depth of field is extended using a phase mask so as to simultaneously provide a bright image and an extended depth of field for wavelength ranges other than red colors (i.e., blue and green colors). This enables observation of fine structure of the blood vessel structure running in the depth direction of a near-surface layer of living body tissue.

In more detail, for the wavelength ranges (i.e., blue and green colors) where the pupil is enlarged so as to obtain fine details of an object of interest and thus high contrast, an optical phase mask is placed in the imaging optical system so as to perform a spatial frequency property conversion, thereby making the optical transfer function almost constant even if the object deviates from the ideal in-focus position. For an image in a wavelength band where the optical transfer function has become almost constant, if signal processing is then performed to restore the spatial frequency content, the depth of field can be dramatically extended.

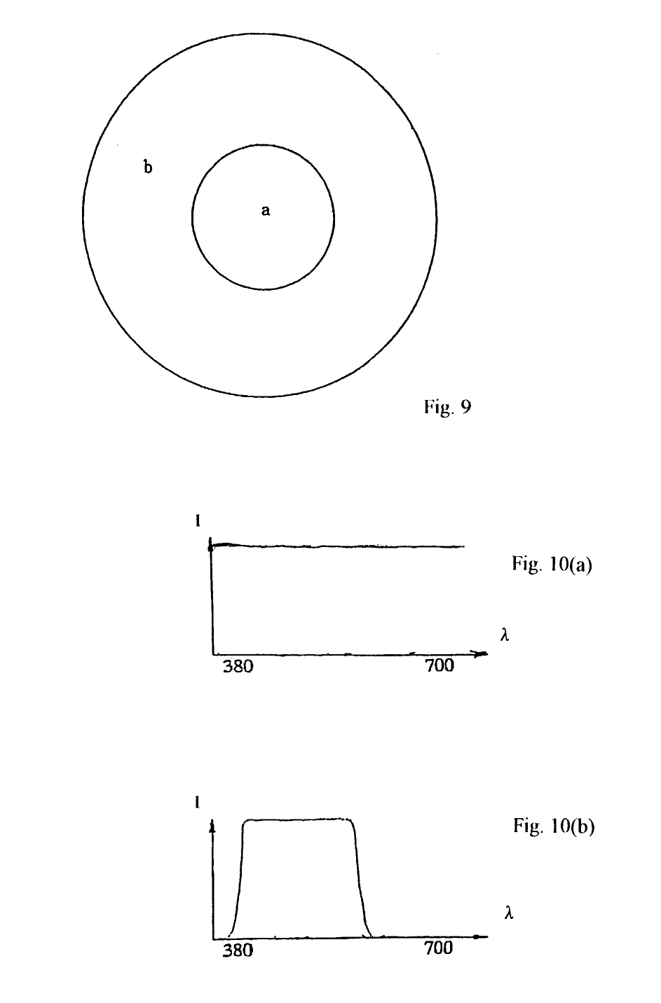

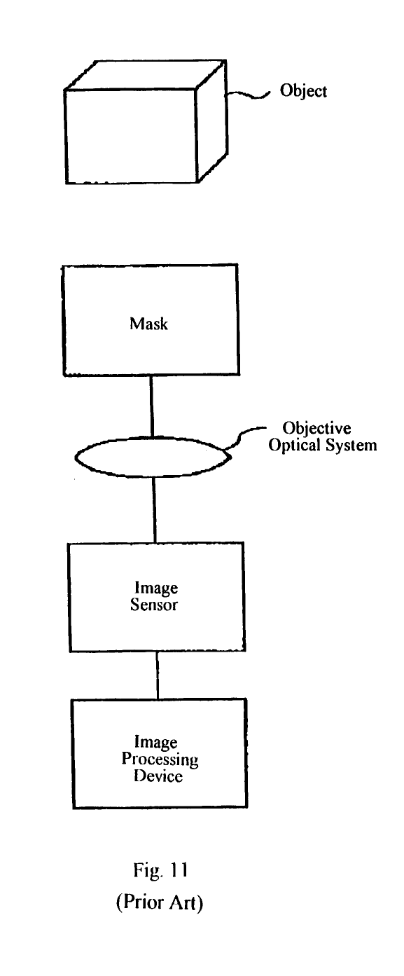

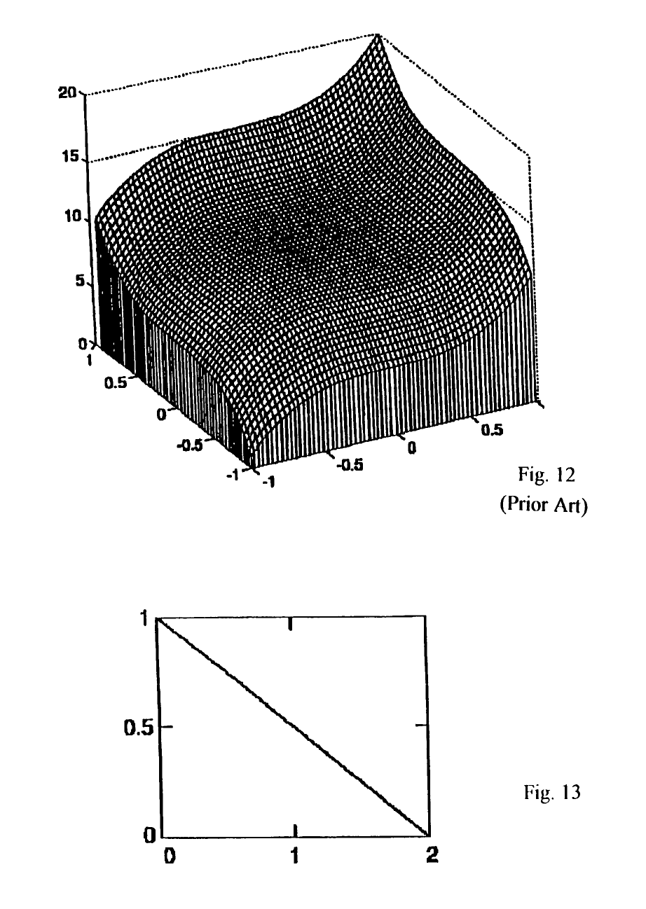

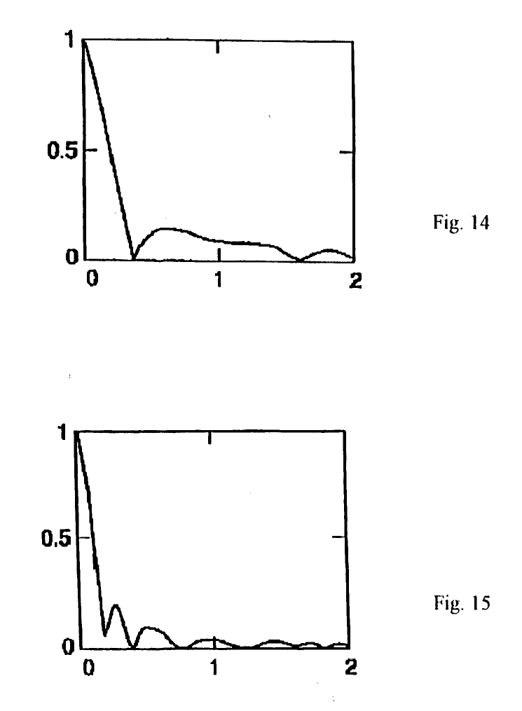

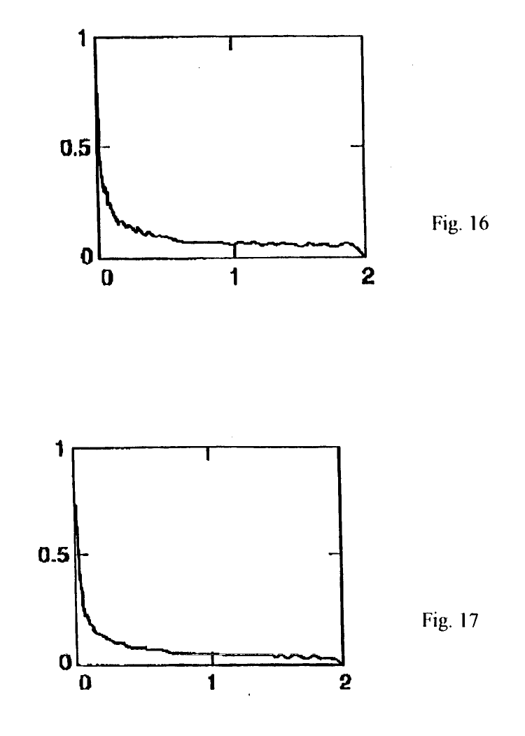

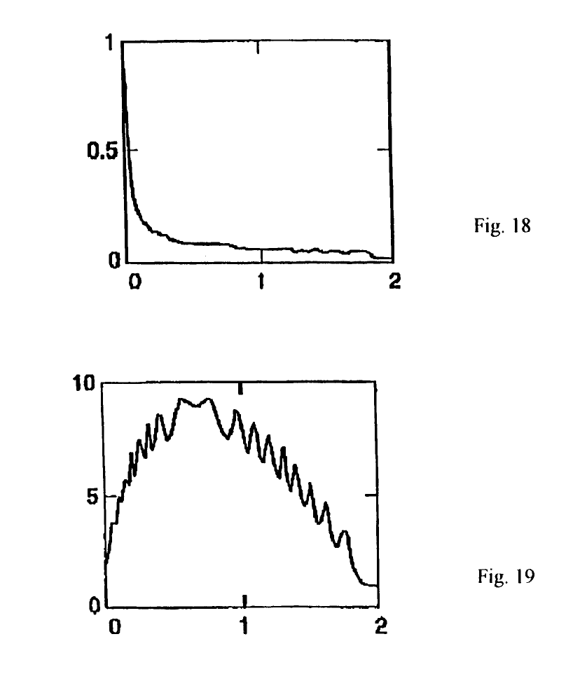

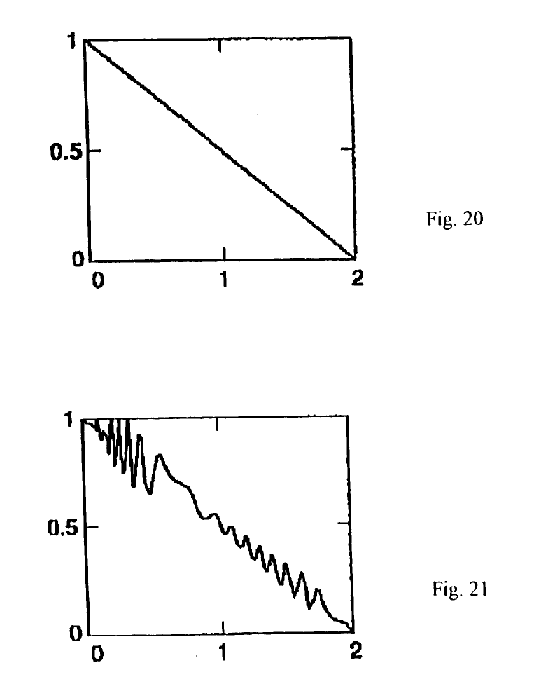

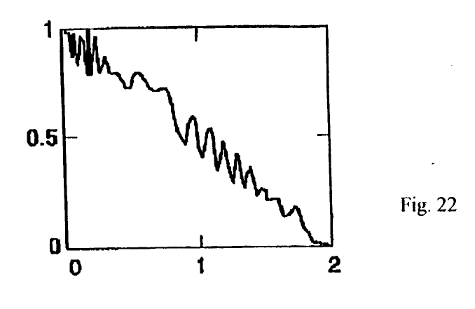

In living body tissues, especially in early cancerous lesion parts, changes unique to cancer appear in the structure of capillary blood vessels that are distributed in the surface layer of living body mucosa. If the mucosa surface layer is magnified using an objective optical system of high magnification, the capillary blood vessels distributed in the mucosa surface layer may be observed using blue light. If the pupil of the imaging optical system is enlarged for the blue color wavelength bands, for example by using a spectral filter as per FIG. 9, observation of the living body mucosa surface layer becomes possible even if the object deviates from the ideal “in-focus” position.

For an image in a wavelength band where the optical transfer function has become almost constant, if signal processing to restore the spatial frequency content is then performed so as to extend the depth of field, tissue information whose contrast is increased can be viewed without blurring over an extended depth of field, which allows the images to be magnified so as to provide an effective endoscope observation.

Also, by making the spectral properties of the filters in the R, G and B wavelength regions such that the wavelength passbands do not overlap, and by using narrow-band light that is sequentially irradiated onto an object of interest, it becomes possible to visually inspect the diseased area.

It is best to increase the depth of field of an observation by performing a depth of field extension technique in the wavelength band where the pupil is enlarged. For observing auto fluorescence of a living body, or for observing the fluorescence from a chemical that has been injected into a living body, the fluorescent light wavelength band to monitor is specified by the excitation wavelength band.

In the case of detecting fluorescence of living body tissue, reflected light that is scattered back to the detector becomes noise. Because the fluorescence signals are weak, there are many cases where the object must be placed near the objective of the imaging optical system. Furthermore, in order to observe fluorescence signals with a good contrast, the pupil should be enlarged only in the wavelength band of the fluorescence. This is accomplished using a spectral filter near the pupil of the optical system. Thus, it is effective if the above-described, prior art technique using a phase filter followed by spatial frequency restoration is employed to extend the depth of field.

Various embodiments of the present invention will now be described in detail, with reference to the drawings.

### Embodiment 1

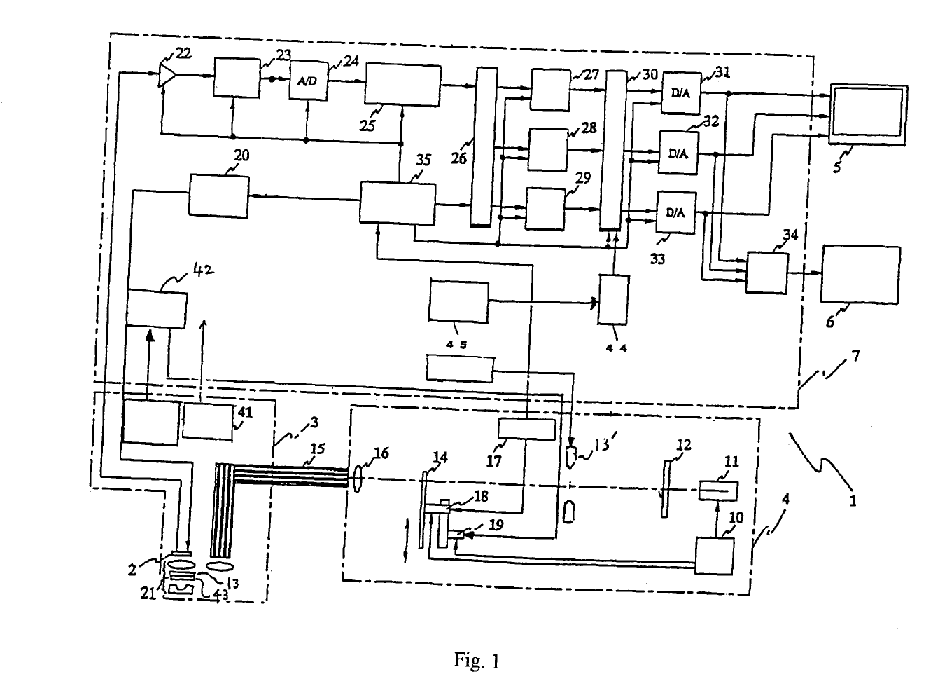

As shown in FIG. 1, the endoscope 1 in this embodiment includes an electronic endoscope 3 having an optical system 21 which is inserted into a coelom for forming images of tissues therein onto a color sensing chip 2 (in this case, the detecting array of the color sensing chip 2 is a CCD), a light source unit 4 which supplies illumination light to the electronic endoscope 3, a video processor 7 which is used to process image signals received from the color sensing chip 2 so as to display images on an observation monitor 5. Also, the endoscope image may be coded and output to a digital filing device 6.

The light source unit 4 is equipped with a xenon lamp 11 which emits illumination light, an infrared-blockinig filter 12 which shields infrared rays of the xenon lamp 11, a diaphragm unit 13′ which limits the amount of the visible light that passes through to a rotating filter wheel 14, and a control circuit 17 which controls the rotation of the rotating filter wheel 14.

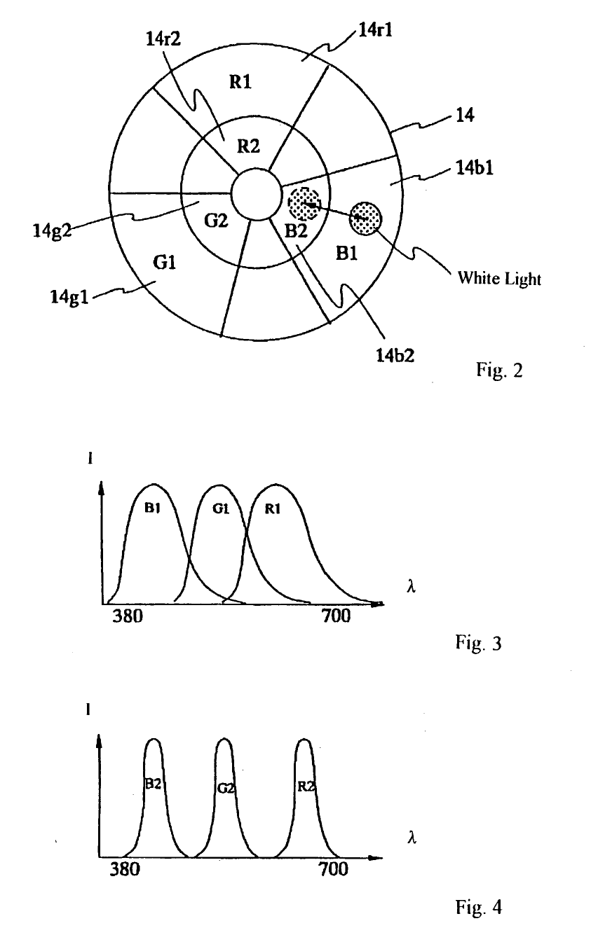

As shown in FIG. 2, the rotating filter wheel 14 is configured with its center as a rotation axis and has a dual structure consisting of an outer part and an inner part. In the outer part portions R1, G1, B1, respective filters 14r1, 14g1, and 14b1 of a first filter set are positioned, as shown. The filters 14r1, 14g1, and 14b1 have overlapping spectral properties (shown in FIG. 3) suitable for color reproduction. In the inner part portions R2, G2, B2, respective filters 14r2, 14g2, and 14b2 of a second filter set are positioned as shown. The filters 14r2, 14g2, and 14b2 have discrete spectral properties (shown in FIG. 4) which enable the extraction of desired deep layer tissue information.

As shown in FIG. 1, the filter wheel 14 is rotated by a rotating filter motor 18 that is controlled by a control circuit 17. Also, movement of the filter wheel 14 so that the inner versus outer portions of the filter wheel are placed in the light path is performed by a mode switching motor 19 according to control signals from a mode switching circuit 42 inside a video processor 7, which will be described later. By this movement, the first filter set or the second filter set of the filter wheel 14 is selectively placed on the optical axis. A xenon lamp 11, the diaphragm unit 13′, the rotating filter motor 18, and the mode switching motor 19 are provided with electric power from a power supply unit 10.

The video processor 7 is equipped with a CCD driver 20 which drives the color sensing chip 2, an amplifier 22 which amplifies image signals taken of tissues inside a coelom using the color sensing chip 2 via an optical system 21, a processing circuit 23 which performs correlated double sampling, noise removal, etc. to the imaging signals that went through the amplifier 22, an A/D converter 24 which converts an imaging signal that passed through the processing circuit 23 to image data of digital signals, an image processing circuit 30 which reads each image data of the sequential light and performs a correction process, such as an outline-emphasizing process or a color processing, etc., D/A circuits 31, 32, and 33 which convert the image data from the image processing circuit 30 to analog signals, an encoding circuit 34 which encodes outputs of the D/A circuits 31, 32, and 33, and a timing generator 35 which inputs a synchronizing signal synchronized with the rotation of the filter wheel and outputs various kinds of timing signals to the circuits. Also, the video processor 7 is designed so that a plural number of electronic endoscopes can be connected.

At least one of the plural number of electronic endoscopes 3, has inside its optical system 21 a spatial frequency property conversion means 13 such as an optical phase mask. Installed in the pupil 43 of the optical system 21 is a spectral filter which has an effective F-number that depends on the wavelength of the light that is transmitted by the spectral filter. Installed in the video processor 7 is an image processing circuit 30 that serves as a spatial frequency restoration means. A reverse frequency property filter (which corresponds to the spatial frequency property of each wavelength band of R, G, B), or program data (which include formulae and numerical values of a corresponding digital filter) is transferred to the image processing circuit 30 from a memory 44 where they are stored, and a spatial frequency property restoration processing is performed to the images obtained using the electronic endoscope 3. Also, in order to distinguish the type of electronic endoscope that is connected, a distinguishing circuit 41 is installed in the electronic endoscope 3, and a control circuit 45 is installed in the video processor 7.

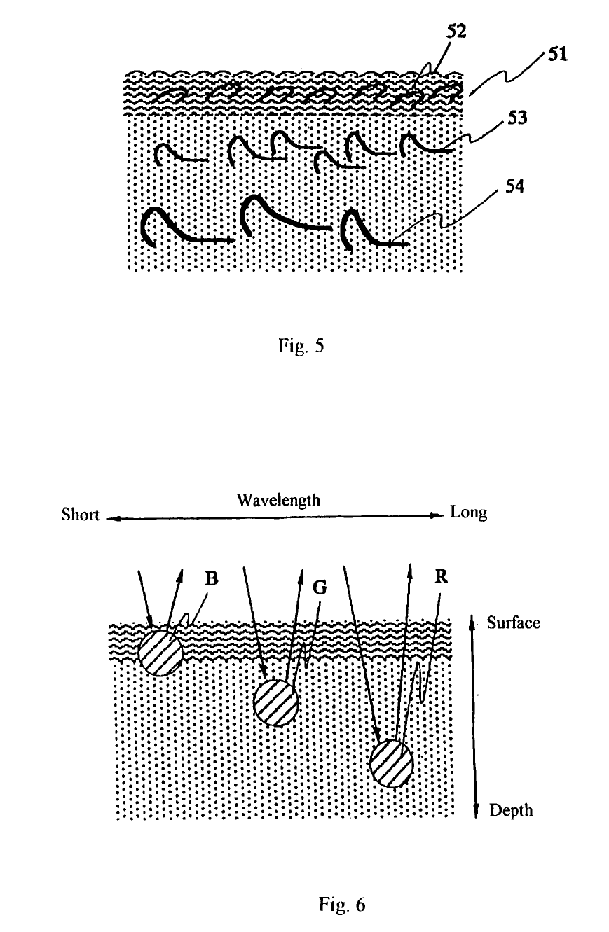

An explanation will now be given on the operation of the endoscope of the invention. As described above, tissues in the coelom have a structure such as that shown in FIG. 5. On the other hand, the penetration depth of light to tissues in a coelom 51 depends on the wavelength of the light. As shown in FIG. 6, illumination light containing the visible range only reaches near the surface layer due to the absorption properties and scattering properties of living tissue. When the illumination light has a short wavelength, such blue light (B), it is absorbed and scattered in a range near the surface, and the scattered light which then emerges from the surface is observed. Also, as is know in the art, green light (G) reaches a deeper range than the range where blue light reaches, is absorbed and scattered in that deeper range, and the light which emerges from the surface is then observed. In the case of red light (R), it reaches a still deeper range, and the light which emerges from the surface is then observed.

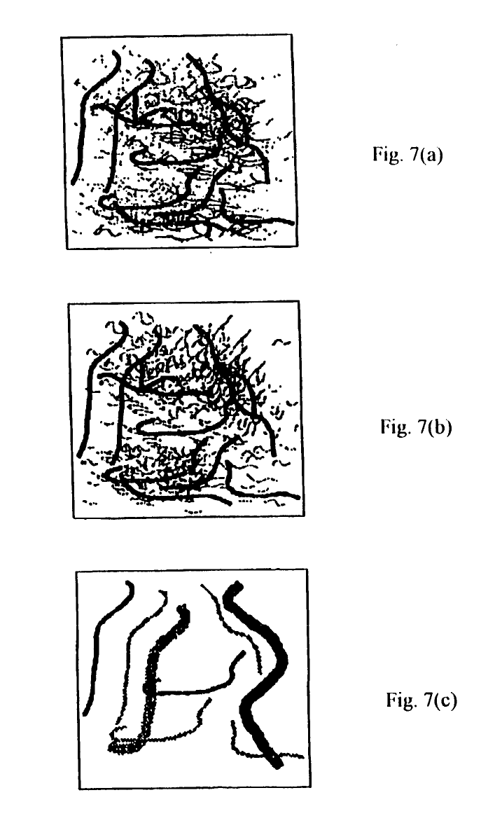

During ordinary observation, the mode switching circuit 42 (FIG. 1) in the video processor 7 controls the mode switching motor 19 using a control signal so that the first filter set formed of filters 14r1, 14g1, and 14b1 (FIG. 2) are positioned in the optical path of the illumination light. The spectral properties of the first filter set are such that, because their wavelength ranges are overlapped as shown in FIG. 3, the filter 14b1 provides an image of shallow layer and middle layer tissues with the image containing a large amount of tissue information for the shallow layer, as shown in FIG. 7(a). The filter 14g1 provides an image of shallow layer and middle layer tissues, with the image containing a large amount of tissue information for the middle layer, as shown in FIG. 7(b). The filter 14r1 provides an image of middle layer and deep layer tissues, with the image containing a large amount of tissue information for the deep layer, as shown in FIG. 7(c).

Referring to FIG. 1, a transmittance filter having two sections, which forms the light beam illustrated in FIG. 9, is positioned at pupil 43 which is immediately adjacent the brightness diaphragm of the optical system 21 of the connected electronic endoscope 3. The center section of the transmittance filter which forms the central region “a” of the light beam has a spectral transmittance as shown in FIG. 10(a), and the peripheral section of the transmittance filter which forms the peripheral region “b” of the light beam has a spectral transmittance as shown in FIG. 10(b). Therefore, the F-number of the transmittance filter varies, depending on the wavelength band transmitted. For red light, the F-number of the transmittance filter is large (i.e., the numerical aperture small), and for blue light and green light the F-number of the transmittance filter is small (i.e., the numerical aperture is large). This enables those spectral portions of an image that contain information of special interest to be imaged with higher contrast. Thus, in the situation illustrated, blue light and green light images will have finer details in the image and thus higher contrast than red light images. The border between the two sections of the transmittance filter need not be circular, and the band of light that is transmitted with a low F-number may be set for any wavelength band the contrast of which is desired to be increased.

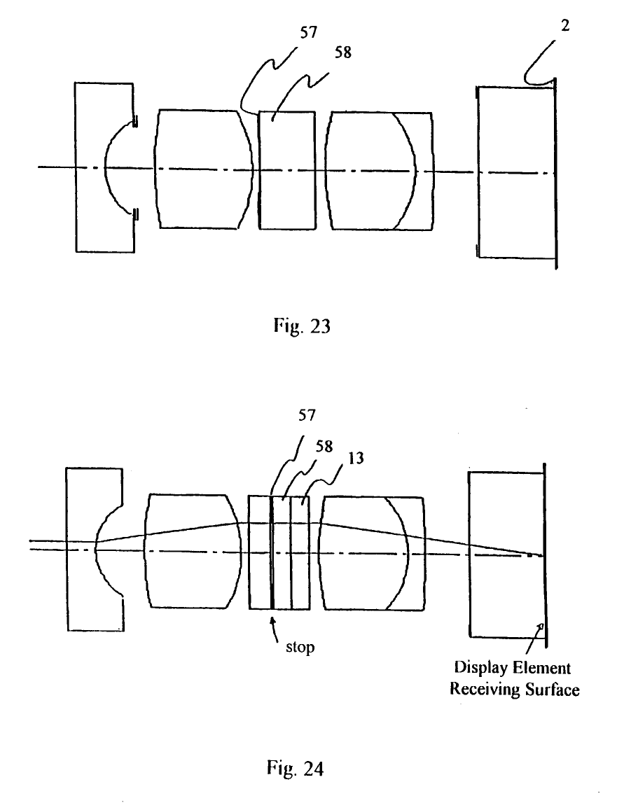

FIG. 23 shows a cross-sectional view of an optical system according to the invention. Immediately after the aperture stop 57 of the optical system a spectral filter 58 is provided. The spectral filter is formed of thin films positioned at a planar surface, with the thin films arranged as per FIG. 9. The spectral transmittance of the regions “a” and “b” shown in FIG. 9, is as illustrated in FIGS. 10(a) and 10(b), respectively. The spectral filter 58 may instead be positioned before or at the aperture stop, and the regions “a” and “b” need not be as illustrated. Moreover, so long as a spectral filter having a transmittance distribution that varies in the radial direction of a color sensing chip is used, by synchronizing and processing RGB image signals using the video processor 7, a desired image such as an endoscope image or an image with natural color reproduction can be obtained having increased spatial frequency content for certain wavelengths.

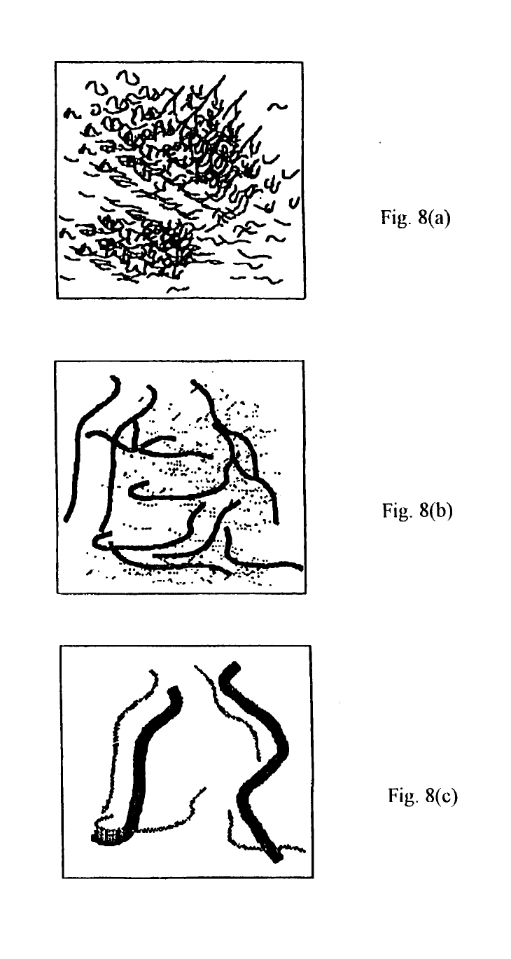

By switching modes of the filter wheel 14 of the light source unit 4, the first filter set, which is in the light path during ordinary observations, can be moved out of the light path and the second filter set moved into the light path. Because the spectral properties of the second filter set provide narrow-band, sequential passbands as shown in FIG. 4, band images providing information of tissue in the shallow layer as shown in FIG. 8(a) may be imaged via the B2 portion filter 14b2, band images providing information of tissue in the middle layer as shown in FIG. 8(b) may be imaged via the G2 portion filter 14g2, and band images providing information of tissue in the deep layer as shown in FIG. 8(c) may be imaged via the R2 portion filter 14r2.

As described above, in this embodiment during the ordinary observation of tissues in a coelom, by shifting to narrow-band observation (such as by switching from the first filter set to the second filter set of the filter wheel 14), tissue information of each layer of tissues in the coelom can be obtained under each separate condition. Namely, deep part information can be viewed with high contrast by using the first filter set, and information on only a specific deep part as a target may be viewed with high contrast by switching to the second filter set.

### Embodiment 2

Concerning Embodiment 2, only the points that are different from Embodiment 1 will be discussed herein, as like items have been labeled the same as for Embodiment 1. FIG. 24 is a cross-sectional view of the optical system of this embodiment. A spectral filter 58 having an inner region “a” and an outer region “b” similar to that of FIG. 9 is placed immediately after an aperture stop 57 of the optical system. The spectral filter 58 has a spectral transmittance in region (b) as shown in FIG. 10(b), so that the effective F-number of the spectral filter in wavelength bands other than red are smaller than the effective F-number of the color spectral filter chip in the red wavelength band. Behind the spectral filter 58, a spatial frequency property conversion means 13 such as a pupil modulation element formed of an optical phase mask is installed. By converting the spatial frequency property, in the wavelength band shown in FIG. 10(b) where the aperture is enlarged, the optical transfer function becomes insensitive to the object distance in a specific range compared to cases where the spatial frequency property is not converted.

For the converted spatial frequencies, by performing a spatial frequency restoration process only to signals for blue light and green light by using an image processing circuit in the video processor 7, the depth of field is enlarged only in the wavelength band where contrast has become higher. By this means, in the blue color band and the green color band where high spatial frequency image components are relatively abundant, high contrast can be realized with a wide depth of field range, and effective images can be provided for observation using an endoscope.

### Embodiment 3

Concerning Embodiment 3, only the points different from that of Embodiment 1 will be discussed herein, as like items have been labeled the same as for Embodiment 1.

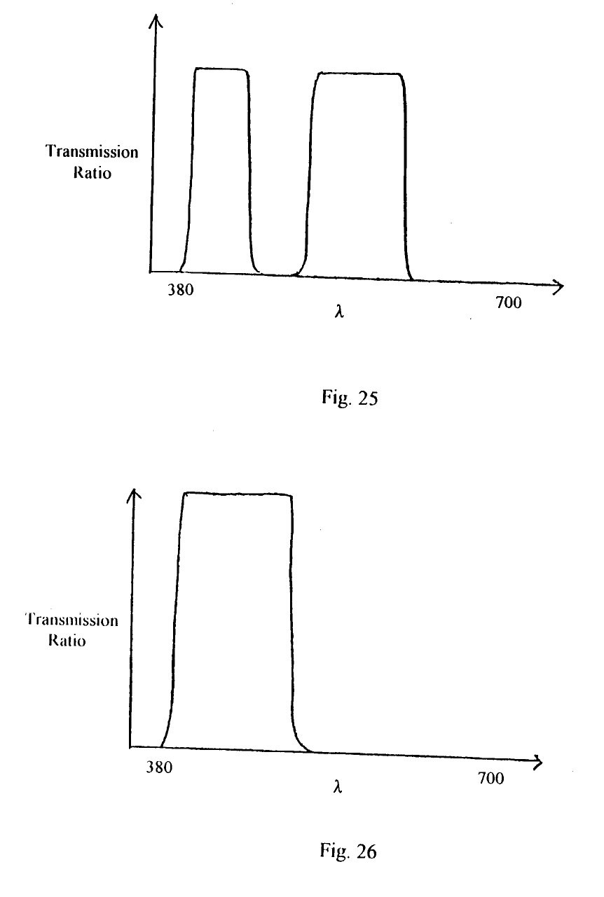

In the optical system of this embodiment, the spectral transmittance in the region where the pupil is enlarged includes the blue and green wavelength bands, as shown in FIG. 25. By this means, when diagnosing a cancerous lesion located near the surface layer of a living body tissue where blue light is scattered/absorbed, as well as in a slightly deeper part than the surface layer of a living body where green light is scattered/absorbed, high-contrast and bright observation of such a lesion becomes possible for observation using an endoscope.

### Embodiment 4

Concerning Embodiment 4, only the points different from that of Embodiment 1 will be discussed herein, as like items have been labeled the same as for Embodiment 1. In the optical system of this embodiment, the spectral transmittance in the region where the pupil is enlarged is shown in FIG. 26. As seen from this figure, the pupil is enlarged only in the blue light band. Thus, the image contrast is increased only in the blue light band. Behind the spectral filter 58, a spatial frequency property conversion means 13, such as a pupil modulation element that serves as an optical phase mask is installed. By converting the spatial frequency property in the wavelength band shown in FIG. 26 where the aperture is enlarged, the optical transfer function becomes insensitive to the object distance in a specified range as compared with the case where the spatial frequency property is not converted. For the converted spatial frequency properties, by performing spatial frequency restoration processing only to signals for blue light by an image processing circuit in the video processor 7, depth of field is increased only in the wavelength band where contrast has become higher. By this means, in the blue color band where high frequency components are relatively abundant, high contrast can be realized over a wide depth of field range, and an effective image can be provided for observation using an endoscope.

### Embodiment 5

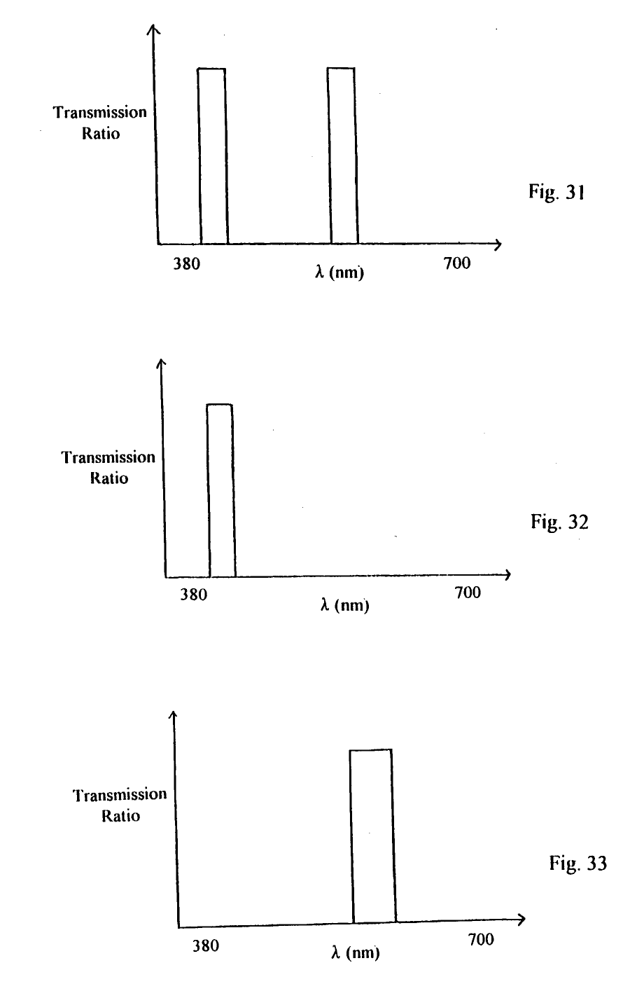

Concerning Embodiment 5, only the points different from that of Embodiment 1 will be discussed herein, as like items have been labeled the same as for Embodiment 1. In the optical system of this embodiment, the transmission ratio versus wavelength in the region “b” of the spectral filter is as shown in FIG. 31. Namely, the region “b” of the spectral filter transmits wavelengths λ in the ranges:

400 nm≦λ≦430 nm, and

550 nm≦λ≦580 nm.

By this means, the structure of capillary blood vessels distributed in the surface layer of a living mucosa and the structures of capillary blood vessels as well as blood vessels thicker than capillary blood vessels in a middle layer that lies deeper than this surface layer can be viewed with high contrast.

### Embodiment 6

Concerning Embodiment 6, only the points different from that of Embodiment 1 will be discussed herein, as like items have been labeled the same as for Embodiment 1. In the optical system of this embodiment, the transmittance ratio versus wavelength in the region “b” (FIG. 9) where the pupil is enlarged is as shown in FIG. 32. In other words, the enlarged area of the pupil transmits for wavelengths λ in the range:

400 nm≦λ≦430 nm.

By this means, the structure of capillary blood vessels distributed in the surface layer of a living mucosa can be imaged with high contrast.

### Embodiment 7

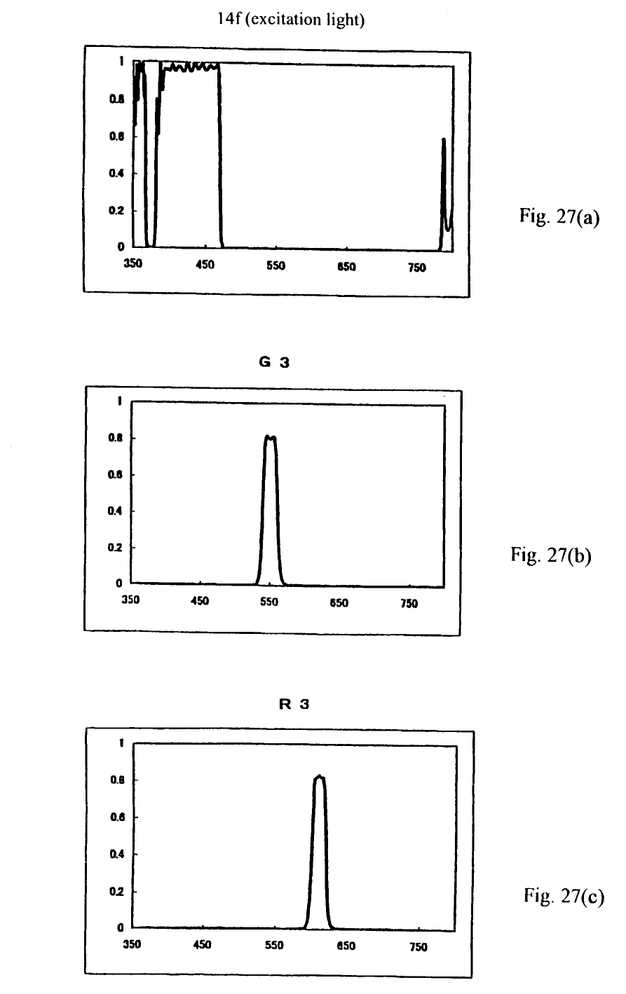

Concerning Embodiment 7, only the points different from that of Embodiment 1 will be discussed herein, as like items have been labeled the same as for Embodiment 1. In the light source unit 4 of this embodiment, in place of the filters 14r2, 14g2, and 14b2, the second filter set consists of a filter 14f having a spectral transmission in the B2 portion as illustrated in FIG. 27(a) which serves for excitation of fluorescence, and the filters in the G2 portion and R2 portion have spectral transmissions G3 and R3, as shown in FIGS. 27(b) and 27(c), respectively.

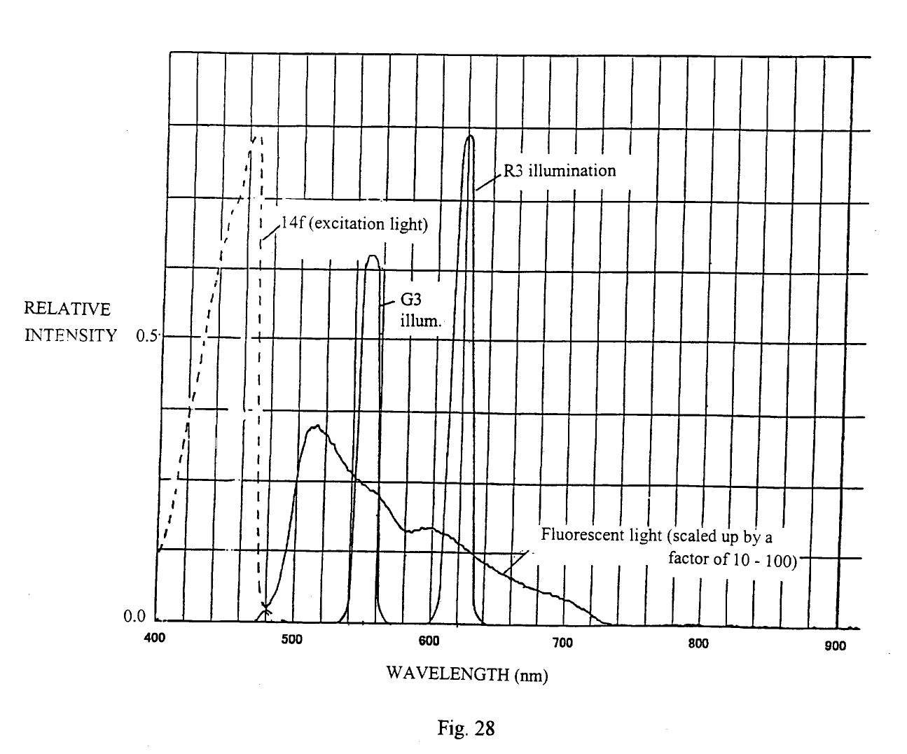

The intensity distribution of reflected light and fluorescent light from an object when illuminated using this second filter set is shown in FIG. 28. When living body tissue is irradiated with the narrow-band excitation light by the filter 14f, fluorescence at the wavelengths shown in FIG. 28 is emitted from the living body tissue. Note that the amount of the fluorescent light is extremely weak, being 1/10 to 1/100 that of light reflected by living tissue when illuminated by the filter 14f, or by the filter which yields the spectrum G3, or by the filter which yields the spectrum R3. Thus, in FIG. 28, the intensity of the fluorescent light is scaled up by a factor or 10-100. Because fluorescent light observation distinguishes tumor portions and normal portions by using brightness, in order to be able to observe the range of the tumor portion precisely, the spectral filter in the optical system of this embodiment has an array of filters in the region “b” portion of the light beam, as shown in FIG. 9, the spectral transmittance of which is shown in FIGS. 27(b) and FIG. 27(c) so that the effective F-numbers in the wavelength bands where fluorescent light is emitted become small. Behind the spectral filter 58 is installed a spatial frequency property conversion means 13 such as a pupil modulation chip which is an optical phase mask. By converting the spatial frequency property, in the wavelength band shown in FIG. 10(b) where the aperture is enlarged, the optical transfer function becomes insensitive to the object distance in a specified range as compared with the case where the spatial frequency property is not converted. For this converted spatial frequency property, by performing a spatial frequency restoration processing by an image processing circuit in the video processor 7 for signals in the wavelength band where the pupil is enlarged, the depth of field increases. By this means observations are made easier, and it is very effective when specifying the boundary between a tumor portion and a normal portion.

Also, the spectral filter may be one that changes its spectral transmittance distribution three times, namely, during the period when the fluorescent light is received, during the period when reflected light of G3 illumination is received, and during the period when reflected light of R3 illumination is received. In the period when fluorescent light is received, the spectral filter is equipped with a spectral transmittance distribution such that a low effective F-number for light in the fluorescent wavelength band is achieved and reflected light of the filter 14f is blocked.

In the period when the reflected light of G3 and R3 illumination is received, as illustrated in FIGS. 27(b) and 27(c) respectively, the spectral filter is equipped in the enlarged region with a spectral transmittance distribution such that the total amount of light in the wavelength range of reflected light of G3 and R3 in FIG. 28 is reduced by a factor of about 10 to 100. By this means, it becomes possible to obtain a color image where the range of a tumor is displayed brightly and clearly with good contrast against the background by synthesizing the fluorescent light image of part of a tumor with such a reduced background created using the reflected light of G3 and R3.

### Embodiment 8

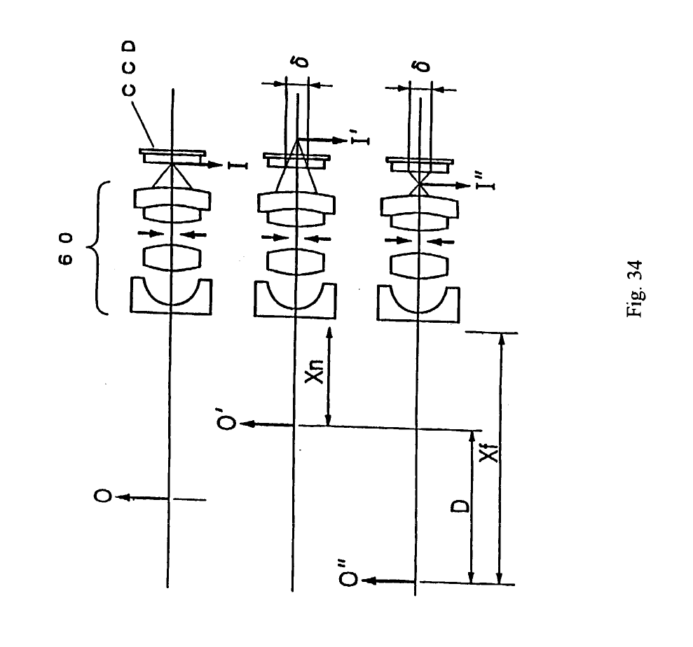

Concerning Embodiment 8, only the points different from that of Embodiment 1 will be discussed herein, as like items have been labeled the same as for Embodiment 1. In the optical system of this embodiment, the spectral transmittance in the wavelength λ region where the pupil is enlarged is as shown in FIG. 33, namely, in the range 550 nm≦λ≦600 nm. By this means, fluorescent light images can be extracted efficiently.

### Embodiment 9

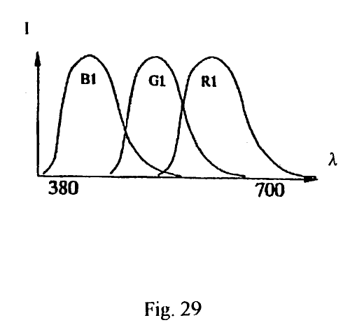

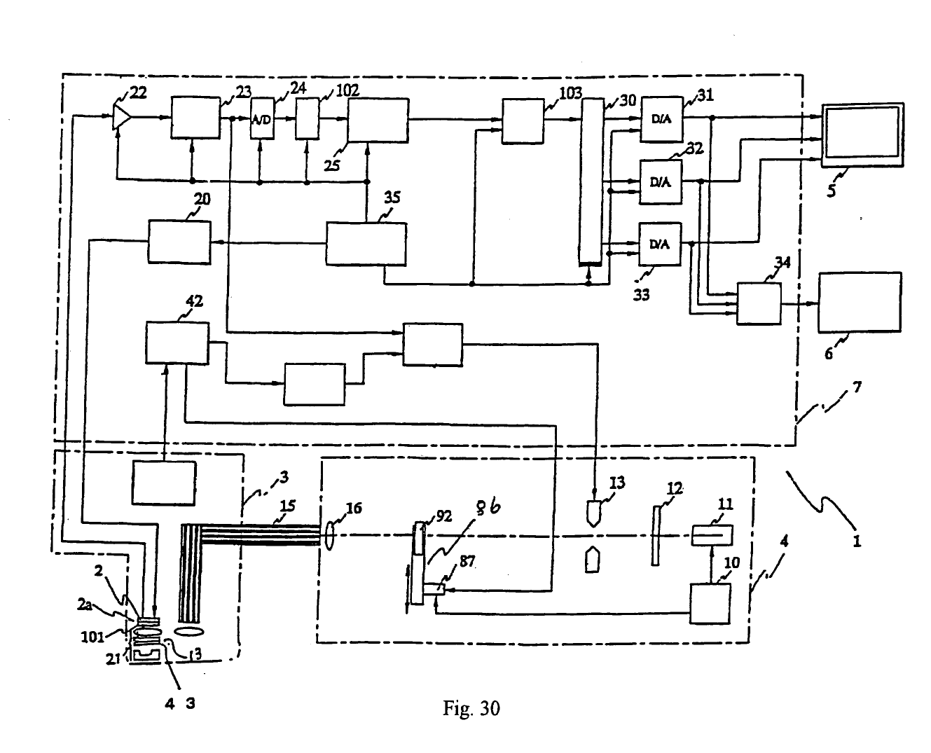

In Embodiment 9, during ordinary observation, filter disc 86 is removed from the light path and white light is irradiated onto a living body tissue. Then, images of the living body tissue illuminated by white light are taken with a color CCD 2a. The spectral properties of the filters in a spectral filter array 101 in front of a CCD are shown in FIG. 29. As shown in FIG. 30, in the electronic endoscope 3 of this embodiment, a spectral filter array 101 is placed on the front surface of a CCD 2 to convert it to a color CCD 2a, constituting a synchronous endoscope 1. Color image signals from the color CCD 2a, after being converted to color image data with an A/D converter 24, are color decomposed by a color separation circuit 102, input to a white balancing circuit 25, and stored in a memory 103. Subsequently, interpolation processing, etc., is performed by an imag processing circuit 30, and then the desired image processing is performed. The filters used with the array of a color sensing chip in the optical system 21 have a spectral transmittance distribution as shown in FIG. 10(b) so that the effective F-numbers in the wavelength bands other than red become small.

Before the spectral filter array 101, a spatial frequency property conversion means 13, such as a pupil modulation element that serves as an optical phase mask, is installed. By converting the spatial frequency properties, in the wavelength band shown in FIG. 10(b) where the aperture is enlarged, the optical transfer function becomes insensitive to the object distance in a specified range compared with the case where the spatial frequency property is not converted. For the converted spatial frequency properties, by performing spatial frequency restoration processing only to signals for blue light and green light by an image processing circuit in the video processor 7, depth of field is only enhanced in the wavelength band where the contrast has become higher. By this means, in the blue color band and green color band where high spatial frequency image components are relatively abundant, high contrast can be realized with a wide depth of field range, and an effective image can be provided through endoscope observations.

The invention being thus described, it will be obvious that the same may be varied in many ways. For example, rather than a spectral filter that is divided into regions having different transmittances so as to enlarge the pupil for specified wavelengths of interest, separate spectral filters having different transmittances and different shapes may instead be used so as to accomplish the same function. Such variations are not to be regarded as a departure from the spirit and scope of the invention. Rather, the scope of the invention shall be defined as set forth in the following claims and their legal equivalents. All such modifications as would be obvious to one skilled in the art are intended to be included within the scope of the following claims.

What is claimed is:

1. An endoscope comprising:
a light source unit for illuminating an object; and
an optical system which forms images of the object and includes a spectral filter, said spectral filter including a first region which has a first spectral transmission and a second region which is peripheral to the first region and which has a second spectral transmission that is different from the first spectral transmission, to thereby enable endoscope images of the object to be obtained wherein fine details as carried by high spatial frequencies in the image light of certain wavelengths are emphasized for those wavelengths that are passed by the second region of the spectral filter.

2. The endoscope as set forth in claim 1, and further comprising:
an optical phase mask which is configured and placed so that the optical transfer function of the optical system becomes almost constant irrespective of the object distance in a range of the depth of field of the endoscope for wavelengths passed by the second region of the spectral filter.

3. The endoscope as set forth in claim 1, wherein the second region is transmissive of visible wavelengths other than wavelengths which are perceived as being in the red portion of the visible spectrum.

4. The endoscope as set forth in claim 3, and further comprising:
an optical phase mask which is configured and placed so that the optical transfer function of the optical system becomes almost constant irrespective of the object distance in a usable range of the depth of field of the endoscope for wavelengths passed by the second region of the spectral filter.

5. The endoscope as set forth in claim 1, wherein the illumination light is narrow-band, sequential light beams of red, green, and blue color, the wavelengths of which do not overlap.

6. The endoscope as set forth in claim 5, and further comprising:
an optical phase mask which is configured and placed so that the optical transfer function of the optical system becomes almost constant irrespective of the object distance in a usable range of the depth of field of the endoscope for wavelengths passed by the second region of the spectral filter.

7. The endoscope as set forth in claim 6, wherein the second region passes light in the following ranges:
400 nm≦λ≦430 nm, and
550 nm≦λ≦580 nm, where λ is the wavelength of light passed by the second region.

8. The endoscope as set forth in claim 1, wherein the second region passes light in the visible wavelength bands of blue color.

9. The endoscope as set forth in claim 8, and further comprising:
an optical phase mask which is configured and placed so that the optical transfer function of the optical system becomes almost constant irrespective of the object distance in a range of the depth of field of the endoscope for wavelengths passed by the second region of the spectral filter.

10. The endoscope as set forth in claim 9, wherein the illumination light is narrow-band, sequential light beams of red, green, and blue color, the wavelengths of which do not overlap.

11. The endoscope as set forth in claim 4, wherein the illumination light is narrow-band, sequential light beams of red, green, and blue color, the wavelengths of which do not overlap.

12. The endoscope as set forth in claim 10, wherein the second region passes wavelengths λ in the range given by:
400 nm≦λ≦430 nm.

13. An endoscope comprising:
a light source unit for irradiating the interior of a living body with excitation light in a wavelength range which causes self fluorescence of photosensitive materials or coelom tissues;
an optical system which forms images of an irradiated object and includes a spectral filter, said spectral filter including a first region which has a first spectral transmission and a second region which is peripheral to the first region and which has a second spectral transmission that is different from the first spectral transmission, to thereby enable endoscope images of the object to be obtained wherein fine details as carried by high spatial frequencies in the image light of certain wavelengths are emphasized for those wavelengths that are passed by the second region of the spectral filter, the second region having a spectral transmission in the wavelength range of said self-fluorescence.

14. The endoscope as set forth in claim 13, and further comprising:
an optical phase mask which is configured and placed so that the optical transfer function of the optical system becomes almost constant irrespective of the object distance in a range of the depth of field of the endoscope for wavelengths passed by the second region of the spectral filter.

15. The endoscope in the claim 14, wherein the wavelengths λ passed by the second region includes the wavelength band of
550 nm≦λ≦600 nm.

16. An endoscope comprising:
a light source unit for illuminating an object with visible light;
an optical system which forms images of returned light from the object when illuminated by the light source and includes a spectral filter, said spectral filter including a first region which has a first spectral transmission and a second region which is peripheral to the first region and which has a second spectral transmission that is different from the first spectral transmission, to thereby enable endoscope images of the object to be obtained wherein fine details as carried by high spatial frequencies in the image light of certain wavelengths are emphasized for those wavelengths that are passed by the second region of the spectral filter;
an optical phase mask which is configured and placed so that the optical transfer function of the optical system becomes almost constant irrespective of the object distance in a usable range of the depth of field of the endoscope for wavelengths passed by the second region of the spectral filter; and
a signal processing means which reverses the change of the optical transfer function performed by the optical phase mask, to thereby enhance the depth of field in the wavelength band passed by the second region of the spectral filter.

17. The endoscope as set forth in claim 16, wherein the second region of the spectral filter passes visible light other than wavelengths which are perceived as being red.

18. The endoscope as set forth in claim 16, wherein the illumination light is narrow-band, sequential light beams of red, green, and blue color, the wavelengths of which do not overlap.

19. The endoscope as set forth in claim 16, wherein the second region of the spectral filter passes visible light of blue color.

20. The endoscope as set forth in claim 11, wherein the second region of the spectral filter passes wavelengths λ in the following ranges:
400 nm≦λ≦430 nm, and
550 nm≦λ≦580 nm.

21. The endoscope as set forth in claim 6, wherein the second region of the spectral filter passes wavelengths λ in the range
400 nm≦λ≦430 nm.
